SEQUENCE LISTING (1 sequence)

SEQ ID NO: 1 - DNA, 16 bp, Artificial sequence
  Synthetic oligonucleotide
ttgatttaatggttgc

(19) United States
(12) Patent Application Publication
(10) Pub. No.: US 2022/0002723 A1
(43) Pub. Date: Jan. 6, 2022

(54) METHODS FOR THE TREATMENT OF SMALL ROUND CELL TUMORS

(71) Applicant: Board of Regents, The University of Texas System, Austin, TX (US)

(72) Inventors: Joseph A. LUDWIG, Houston, TX (US); Brian A. MENEGAZ, Houston, TX (US); Salah-Eddine LAMHAMEDI-CHERRADI, Houston, TX (US)

(73) Assignee: Board of Regents, The University of Texas System, Austin, TX (US)

(21) Appl. No.: 17/293,596

(22) PCT Filed: Nov. 14, 2019
(86) PCT No.: PCT/US2019/061511

§ 371 Date: May 13, 2021

Related U.S. Application Data
(60) Provisional application No. 62/767,458, filed on Nov. 14, 2018.

(51) Int. Cl. C12N 15/113; A61K 45/06; A61P 35/00

(52) U.S. Cl. CPC C12N 15/113; A61P 35/00; A61K 45/06

(57) ABSTRACT

Provided herein are methods for treating small round cell tumor(s) by administering anti-androgen receptor therapy to a subject. The anti-androgen receptor therapy may comprise an AR antisense oligonucleotide and may be administered in combination with an additional anti-cancer agent.

This application claims the benefit of U.S. Provisional Application No. 62/767,458, filed Nov. 14, 2018, the entirety of which is incorporated herein by reference.

### INCORPORATION OF SEQUENCE LISTING

The sequence listing that is contained in the file named “UTFCP1422WO_ST25.txt”, which is 1 KB (as measured in Microsoft Windows®) and was created on Nov. 14, 2019, is filed herewith by electronic submission and is incorporated by reference herein.

### BACKGROUND

### 1. Field

The present invention relates generally to the fields of medicine and immunology. More particularly, it concerns methods for methods of treating soft tissue sarcoma with anti-androgen receptor therapy.

### 2. Description of Related Art

Malignant small round cell tumors are characterized by small, round, relatively undifferentiated cells. They include Ewing's sarcoma, peripheral neuroectodermal tumor, rhabdomyosarcoma, synovial sarcoma, non-Hodgkin's lymphoma, retinoblastoma, neuroblastoma, hepatoblastoma, and nephroblastoma or Wilms' tumor. Other differential diagnoses of small round cell tumors include small cell osteogenic sarcoma, undifferentiated hepatoblastoma, granulocytic sarcoma, and intraabdominal desmoplastic small round cell tumor (DSRCT).

DSRCT is a rare, aggressive soft tissue sarcoma in adolescents and young adults characterized by the reciprocal EWSR1-WT1 t(11;22)(p13:q12) chromosomal translocation that is diagnostic of this tumor. Given a lack of prospective clinical trials due to its rarity and the molecular and morphological similarities to Ewing sarcoma (ES), DSRCT is typically treated with ES-based chemotherapies despite differing both in clinical presentation and prognosis. Only recently have molecular characterization efforts and proteomic profiling identified a number of aberrations that distinguish DSRCT from ES. However, there is an unmet need for therapies to treat DSRCT.

### SUMMARY

In certain embodiments, the present disclosure provides methods for treating small round cell tumor in a subject comprising administering an effective amount of an anti-androgen receptor (anti-AR) therapy to the subject. In particular aspects, the small round cell tumor is desmoplastic small round cell tumor (DSRCT). In some aspects, the subject is human.

In some aspects, the anti-AR therapy comprises an AR antisense oligonucleotide. The AR antisense oligonucleotide can comprise a sequence at least 80%, 85%, 90%, 95%, 96%, 97%, 98%, 99%, or 100% identical to SEQ ID NO:1 (TTGATTTAATGGTTGC). In particular aspects, the AR antisense oligonucleotide is or comprises AZD5312. In some aspects, the AR antisense oligonucleotide is administered intravenously. In specific aspects, the AR antisense oligonucleotide is administered at a dose of 150-900 mg, such as 175, 200, 250, 300, 350, 400, 450, 500, 550, 600, 650, 700, 750, 800, or 850 mg. The AR antisense oligonucleotide may be administered more than once, such as daily, every 2 days, every 3 days, or every 4 days.

In certain aspects, the anti-AR therapy comprises therapy with an androgen receptor antagonist, androgen synthesis inhibitor, or an antigonadotropin. For example, the androgen receptor antagonist can comprise cyproterone acetate, megestrol acetate, chlormadinone acetate, spironolactone, oxendolone, flutamide, bicalutamide, nilutamide, topilutamide, enzalutamide, or apalutamide. In some aspects, the androgen synthesis inhibitor is or comprises ketoconazole, abiraterone acetate, seviteronel, aminoglutethimide, finasteride, dutasteride, epristeride, or alfatradiol. In certain aspects, the antigonadotropin is or comprises leuprorelin, cetrorelix, allylestrenol, chlormadinone acetate, cyproterone acetate, gestonorone caproate, hydroxyprogesterone caproate, medroxyprogesterone acetate, megestrol acetate, or oxendolone. In particular aspects, the anti-AR therapy is selected from the group consisting of enzalutamide (MDV3100), ARN-509, ODM-201, abiraterone acetate, Galeterone (TOK001), orteronel (TAK700) and VT464.

In additional aspects, a method of the embodiments further comprises administering at least one additional anti-cancer therapy. In some aspects, the anti-cancer therapy is an anti-TAZ therapy and/or an anti-EWSR1 therapy, such as a therapy using antisense oligonucleotides. In some aspects, the anti-cancer therapy is or comprises chemotherapy, immunotherapy, surgery, radiotherapy, or biotherapy. In certain aspects, the anti-cancer therapy is administered orally, intravenously, intraperitoneally, intratracheally, intratumorally, intramuscularly, endoscopically, intralesionally, percutaneously, subcutaneously, topically, regionally, or by direct injection or perfusion. In some aspects, the anti-AR therapy and/or at least one additional anti-cancer therapy may be administered simultaneously. In certain aspects, the anti-AR therapy is administered prior to the at least one additional anti-cancer therapy.

Further provided herein is a composition comprising an effective amount of an anti-AR therapy for use in the treatment of a small round cell tumor in a subject. In some aspects, the anti-AR therapy comprises an AR antisense oligonucleotide. The AR antisense oligonucleotide can comprise a sequence at least 80%, 85%, 90%, 91%, 92%, 93%, 94%, 95%, 96%, 97%, 98%, 99%, or 100% identical to SEQ ID NO:1 (TTGATTTAATGGTTGC). In particular aspects, the AR antisense oligonucleotide is AZD5312. In some aspects, the AR antisense oligonucleotide is administered intravenously. In specific aspects, the AR antisense oligonucleotide is administered at a dose of 150-900 mg, such as 175, 200, 250, 300, 350, 400, 450, 500, 550, 600, 650, 700, 750, 800, or 850 mg. The AR antisense oligonucleotide may be administered more than once, such as daily, every 2 days, every 3 days, or every 4 days.

In certain aspects, the anti-AR therapy is an androgen receptor antagonist, androgen synthesis inhibitor, or an antigonadotropin. For example, the androgen receptor antagonist can comprise cyproterone acetate, megestrol acetate, chlormadinone acetate, spironolactone, oxendolone, flutamide, bicalutamide, nilutamide, topilutamide, enzalutamide, or apalutamide. In some aspects, the androgen synthesis inhibitor is ketoconazole, abiraterone acetate, seviteronel, aminoglutethimide, finasteride, dutasteride, epristeride, or alfatradiol. In certain aspects, the antigonadotropin is leuprorelin, cetrorelix, allylestrenol, chlormadinone acetate, cyproterone acetate, gestonorone caproate, hydroxyprogesterone caproate, medroxyprogesterone acetate, megestrol acetate, or oxendolone. In particular aspects, the anti-AR therapy is selected from the group consisting of enzalutamide (MDV3100), ARN-509, ODM-201, abiraterone acetate, Galeterone (TOK001), orteronel (TAK700) and VT464.

Also provided herein is the use of a composition comprising an effective amount of an anti-AR therapy for the treatment of a small round cell tumor in a subject. In some aspects, the anti-AR therapy comprises an AR antisense oligonucleotide. The AR antisense oligonucleotide can comprise a sequence at least 80%, 85%, 90%, 95%, 96%, 97%, 98%, 99%, or 100% identical to SEQ ID NO:1 (TTGATTTAATGGTTGC). In particular aspects, the AR antisense oligonucleotide is AZD5312. In some aspects, the AR antisense oligonucleotide is administered intravenously. In specific aspects, the AR antisense oligonucleotide is administered at a dose of 150-900 mg, such as 175, 200, 250, 300, 350, 400, 450, 500, 550, 600, 650, 700, 750, 800, or 850 mg. The AR antisense oligonucleotide may be administered more than once, such as daily, every 2 days, every 3 days, or every 4 days.

In certain aspects, the anti-AR therapy is an androgen receptor antagonist, androgen synthesis inhibitor, or an antigonadotropin. For example, the androgen receptor antagonist can comprise cyproterone acetate, megestrol acetate, chlormadinone acetate, spironolactone, oxendolone, flutamide, bicalutamide, nilutamide, topilutamide, enzalutamide, or apalutamide. In some aspects, the androgen synthesis inhibitor is ketoconazole, abiraterone acetate, seviteronel, aminoglutethimide, finasteride, dutasteride, epristeride, or alfatradiol. In certain aspects, the antigonadotropin is leuprorelin, cetrorelix, allylestrenol, chlormadinone acetate, cyproterone acetate, gestonorone caproate, hydroxyprogesterone caproate, medroxyprogesterone acetate, megestrol acetate, or oxendolone. In particular aspects, the anti-AR therapy is selected from the group consisting of enzalutamide (MDV3100), ARN-509, ODM-201, abiraterone acetate, Galeterone (TOK001), orteronel (TAK700) and VT464.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein.

FIGS. 1A-1D: Proteomic comparison of DSRCT and Ewing sarcoma samples. RPPA analysis of DSRCT (left bars in panels B & D) and ES (right bars in panels B & D) patient tumors (A & B), validated by western blotting (C) show an upregulation of AR and other proteins differentiating the two diseases. Densitometry confirms expression differences of key proteins (D).

FIGS. 2A-2B: Expression of AR and its coactivators in DSRCT. Western blotting of additional DSRCT tumors (A) and the JN-DSRCT cell line (B) show further elevated expression of AR and its coactivators, NCOA1/2.

FIG. 3: Stimulation of DSRCT proliferation in vitro via AR. When stimulated with testosterone or DHT, the JN-DSRCT and LNCAP prostate cancer cell lines show increased proliferation. TC-71 Ewing sarcoma cells showed no effect by these AR agonists.

FIG. 4: Inhibition of DSRCT proliferation in vitro via AR. Addition of AR ASO results in knockdown of AR expression in JN-DSRCT cells and inhibits proliferation at 7- and 14-days.

FIGS. 5A-5E: In vivo inhibition of AR reduces tumor burden in DSRCT xenograft and PDX. Immunocompromised NSG mice bearing JN-DSRCT xenograft (A), and DSRCT PDX (B) tumors treated with AR ASOs has significantly reduced tumor burden and improved survival compared to control ASO groups (p=0.0097 & p<0.0001, respectively). Proteomic profiling by western blotting (A & B), immunofluorescence (C & D), and immunohistochemistry (E) analyses validate the knockdown of AR expression in DSRCT xenografts and PDX by AR ASO treatment.

### DESCRIPTION OF ILLUSTRATIVE EMBODIMENTS

In the present studies, it was found that androgen receptor (AR) is highly expressed in desmoplastic small round cell tumor (DSRCT) patients and represents a promising therapeutic target. Specifically, in vitro cell proliferation assays and xenografts drug-testing were performed to evaluate the preclinical efficacy of anti-AR therapy, such as AR-based antisense therapy, for DSRCT. Inhibition of AR by anti-AR anti-sense oligonucleotide (ASO) showed a significant decrease in AR expression at 3 days and cellular proliferation at both 7 and 14 days. In addition, DSRCT patient-derived xenografts and JN-DSRCT tumor cells-bearing NSG immunocompromised mice treated with anti-AR ASOs showed considerably reduced tumor burden and improved survival compared to mice in the placebo- and control ASO-treated groups.

Accordingly, in certain embodiments, the present disclosure provides methods for treating small round cell tumors, such as DSRCT, with anti-AR therapy. The anti-AR therapy may be anti-AR antisense therapy, androgen receptor antagonist, androgen synthesis inhibitor, or antigonadotropin. The anti-AR therapy may be combined with additional anti-cancer therapy, such as TAZ or EWSR1-targeted therapy, such as antisense oligonucleotides.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

As used herein, the terms “treat”, “treatment”, “treating”, and the like refer to the process of ameliorating, lessening, or otherwise mitigating the symptoms of a disease or condition in a subject by, for example, administering a therapeutic agent to the subject, or by performing a surgical, clinical, or other medical procedure on the subject.

As used herein, the terms “subject” or “patient” are used interchangeably herein to refer to an individual, e.g., a human or a non-human organism, such as a primate, a mammal, or a vertebrate.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to affect such treatment or prevention of the disease.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

### II. METHODS OF USE

In some embodiments, the present disclosure provides methods for treating or delaying progression of soft tissue sarcoma, such as small round cell tumors, comprising administering an anti-androgen receptor therapy. The small round cell tumor may be Ewing's sarcoma, peripheral neuroectodermal tumor, rhabdomyosarcoma, synovial sarcoma, non-Hodgkin's lymphoma, retinoblastoma, neuroblastoma, hepatoblastoma, and nephroblastoma or Wilms' tumor, small cell osteogenic sarcoma, undifferentiated hepatoblastoma, granulocytic sarcoma, and desmoplastic small round cell tumor (DSRCT). In particular embodiments, the small round cell tumor is DSRCT.

The anti-AR therapy may comprise an antisense oligonucleotide. An oligomeric compound may be “antisense” to a target nucleic acid, meaning that is capable of undergoing hybridization to a target nucleic acid through hydrogen bonding. The antisense oligonucleotide may be a modified oligonucleotide 10 to 30 linked nucleosides in length targeted to AR. The AR target can have a sequence recited in any one of SEQ ID NO: 1 (TTGATTTAATGGTTGC) or a portion thereof or a variant thereof. In certain embodiments, the AR target can have a sequence of known AR splicing variants including, but are not limited to, AR-V1, AR-V2, AR-V3, AR-V4, AR-V5, AR-V6, and AR-V7 (also referred to as AR3), which contain exons 1-3 but lack exons 4-8. AR-V1, AR-V2, AR-V3, AR-V4, AR-V5, AR-V6, or AR-V7. The modified oligonucleotide may comprise a gap segment consisting of 8 linked deoxynucleosides; a 5′ wing segment consisting of five linked nucleosides; and a 3′ wing segment consisting of three linked nucleosides; In particular, the AR antisense oligonucleotide may be AZD5312 (also known as ISIS-AZ1Rx, ISIS-ARRx, AZD-5312, ISIS-560131, ISIS-AR-2.5Rx, and IONIS-AR-2.5Rx), such as described in U.S. Pat. Nos. 9,567,588 and 9,175,291; incorporated by reference in their entirety herein.

The anti-AR therapy may comprise an androgen receptor antagonist, androgen synthesis inhibitor, and/or an antigonadotropin. Exemplary androgen receptor antagonists include but are not limited the steroidal antiandrogens cyproterone acetate, megestrol acetate, chlormadinone acetate, spironolactone, and oxendolone and the nonsteroidal antiandrogens flutamide, bicalutamide, nilutamide, topilutamide, enzalutamide, and apalutamide. Androgen synthesis inhibitors include drugs that directly inhibit the enzymatic biosynthesis of androgens, such as testosterone and/or DHT. Examples include the CYP17A1 inhibitors ketoconazole, abiraterone acetate, and seviteronel, the CYP11A1 (P450scc) inhibitor aminoglutethimide, and the 5α-reductase inhibitors finasteride, dutasteride, epristeride, alfatradiol, and saw palmetto extract (Serenoa repens). A number of other antiandrogens, including cyproterone acetate, spironolactone, medrogestone, flutamide, nilutamide, and bifluranol, may be used to inhibit androgen synthesis. Antigonadotropins include GnRH modulators like leuprorelin (a GnRH agonist) and cetrorelix (a GnRH antagonist), progestogens like allylestrenol, chlormadinone acetate, cyproterone acetate, gestonorone caproate, hydroxyprogesterone caproate, medroxyprogesterone acetate, megestrol acetate, and oxendolone, and estrogens like estradiol, estradiol esters, ethinylestradiol, conjugated estrogens, and diethylstilbestrol. Particular classes of anti-androgenic agents are the second generation anti-hormonal agents such as: enzalutamide (MDV3100), ARN-509, ODM-201, abiraterone acetate, Galeterone (TOK001), orteronel (TAK700) and VT464.

Androgen deprivation therapy that may be utilized include, but are not limited, to orchiectomy (surgical castration), luteinizing hormone-releasing hormone (LHRH) analogs (e.g., leuprolide, goserelin, triptorelin, or histrelin), luteinizing hormone-releasing hormone (LHRH) antagonists (e.g., degarelix and abiraterone), anti-androgens (flutamide, bicalutamide, nilutamide, and enzalutamide), and other androgen-suppressing drugs (e.g., ketoconazole).

A. Combination Therapies

In certain embodiments, the methods provided herein further comprise a step of administering at least one additional therapeutic agent to the subject. All additional therapeutic agents disclosed herein will be administered to a subject according to good clinical practice for each specific composition or therapy, taking into account any potential toxicity, likely side effects, and any other relevant factors.

In certain embodiments, the additional therapy may be immunotherapy, radiation therapy, surgery (e.g., surgical resection of a tumor), chemotherapy, bone marrow transplantation, or a combination of the foregoing. The additional therapy may be targeted therapy. In certain embodiments, the additional therapy is administered before the primary treatment (i.e., as adjuvant therapy). In certain embodiments, the additional therapy is administered after the primary treatment (i.e., as neoadjuvant therapy.

In certain embodiments, the additional therapy comprises an immunotherapy. In certain embodiments, the immunotherapy comprises an immune checkpoint inhibitor.

An anti-AR therapy may be administered before, during, after, or in various combinations relative to an additional cancer therapy. The administrations may be in intervals ranging from concurrently to minutes to days to weeks. In embodiments where the anti-AR therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the anti-AR therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below anti-AR therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaI 1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin;

losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above

2. Radiotherapy

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibodydrug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs inlcude ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, erb b2 and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDLL and/or PDL2. In another embodiment, a PDLL binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDLL or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIV®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin.

B. Pharmaceutical Compositions

In another aspect, provided herein are pharmaceutical compositions and formulations comprising an anti-AR therapy and a pharmaceutically acceptable carrier.

An antisense compound targeted to an androgen receptor nucleic acid can be utilized in pharmaceutical compositions by combining the antisense compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising an antisense compound targeted to an androgen receptor nucleic acid and a pharmaceutically acceptable diluent. In certain embodiments, the pharmaceutically acceptable diluent is water. In certain embodiments, the antisense compound is an antisense oligonucleotide provided herein.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of aqueous solutions, such as normal saline (e.g., 0.9%)and human serum albumin (e.g., 10%). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zinc-protein complexes); and/or non-ionic surfactants such as polyethylene glycol (PEG).

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Androgen Receptor Antisense Therapy

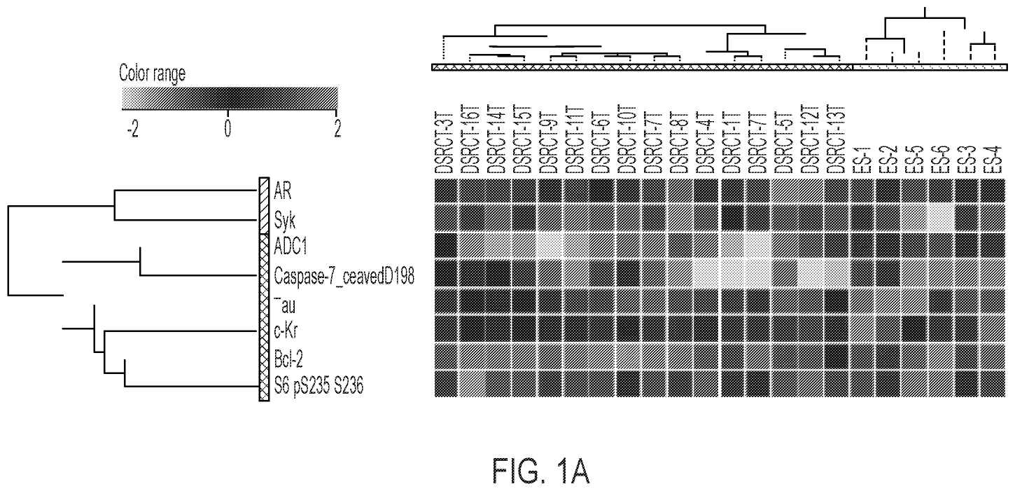

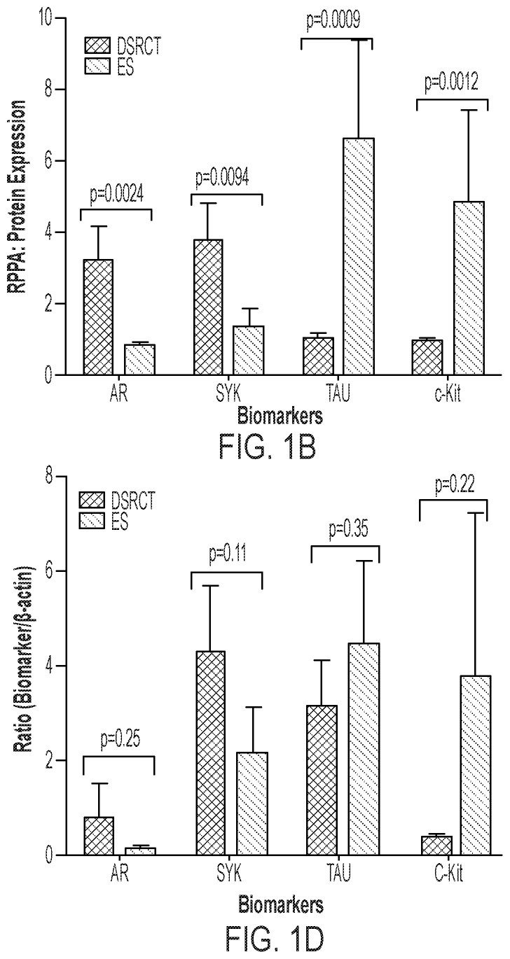

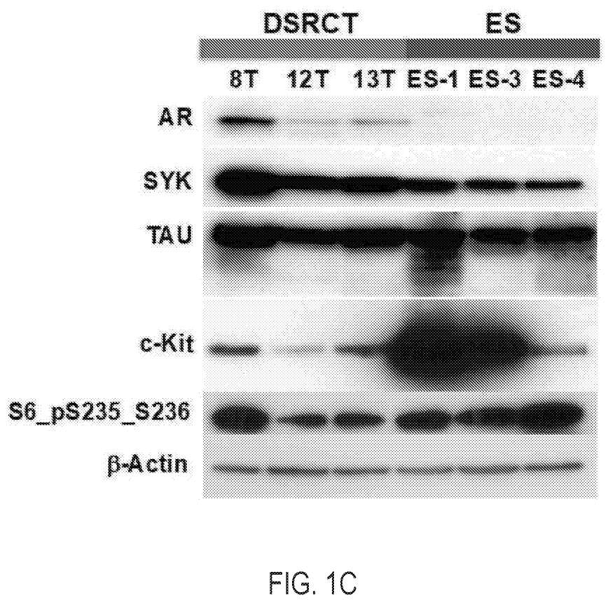

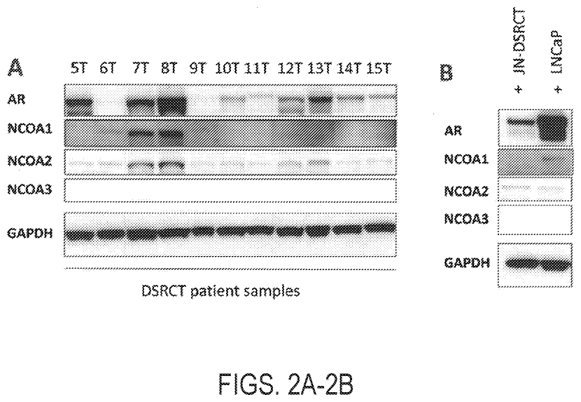

In order to determine whether androgen receptor (AR) may be used as a target for treating small round cell tumors, expression of AR was assessed in small round cells tumors including DSRCT and ES patient samples. Protein lysates were isolated from DSRCT and ES patient tumors, protein concentrations were determined, and individual protein expression was measured using a well-validated reverse-phase protein array (RPPA). Selected proteins that were strongly and consistently upregulated in DSRCT or ES patients were subsequently validated by western blotting (FIG. 1). The JN-DSRCT cell line and DSRCT patient samples expressed high levels of AR as compared to ES samples (FIG. 2).

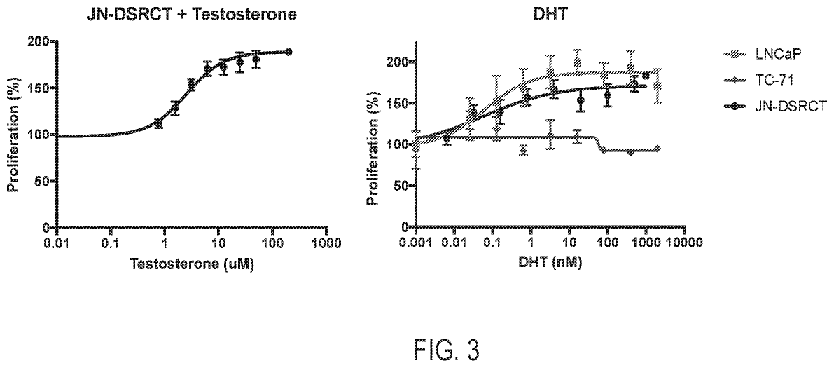

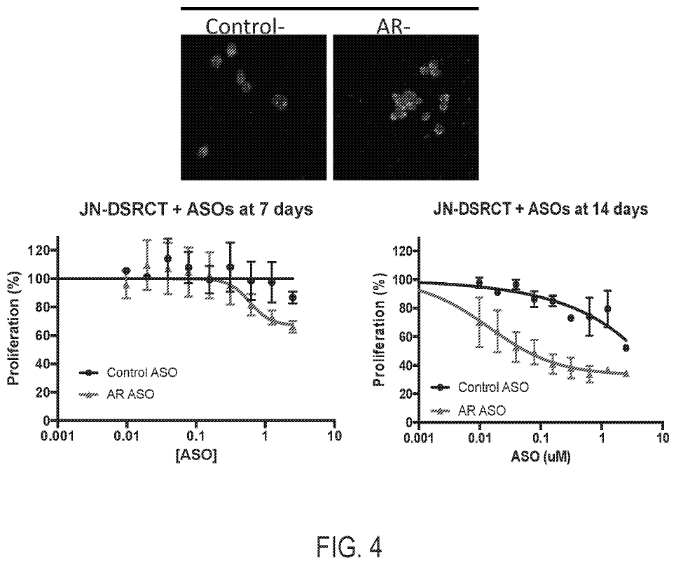

Stimulation of DSRCT, ES TC-71 and prostate cancer LNCaP cell lines with exogenous 5a-dihydrotestosterone (DHT, an AR native ligand) showed increased proliferation in the DSRCT & LNCaP cell lines at high levels of DHT, but not in the TC71 ES cell line which lacks AR expression (FIG. 3). Thus, an increase in AR expression results in increased proliferation of DSRCT cells.

Further, in vitro cell proliferation assays and xenografts drug-testing were performed to evaluate the preclinical efficacy of AR-based anti-sense therapy for DSRCT. It was found that inhibition of AR in the JN-DSRCT cell line by anti-AR anti-sense oligonucleotide (ASO) showed a significant decrease in AR expression at 3 days and cellular proliferation at both 7 and 14 days (FIG. 4). Thus, AR can be used as a target for treating small cell round tumors with high expression of AR, such as DSRCT.

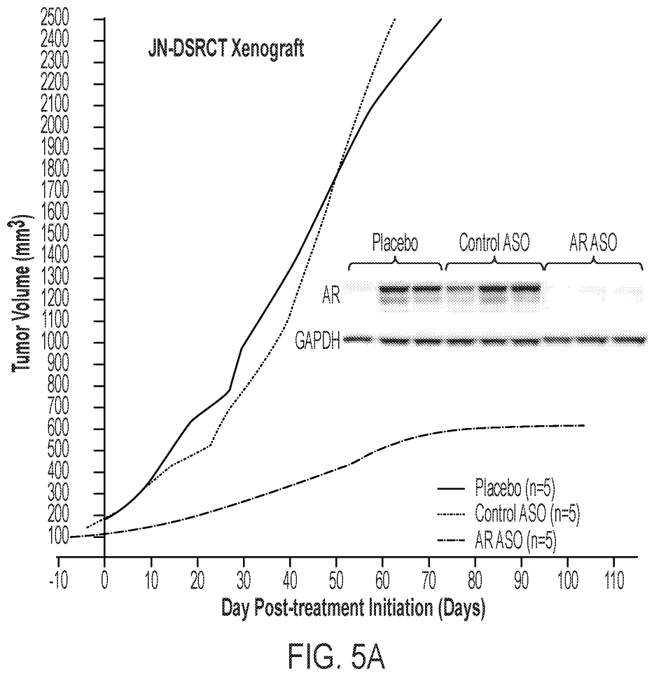

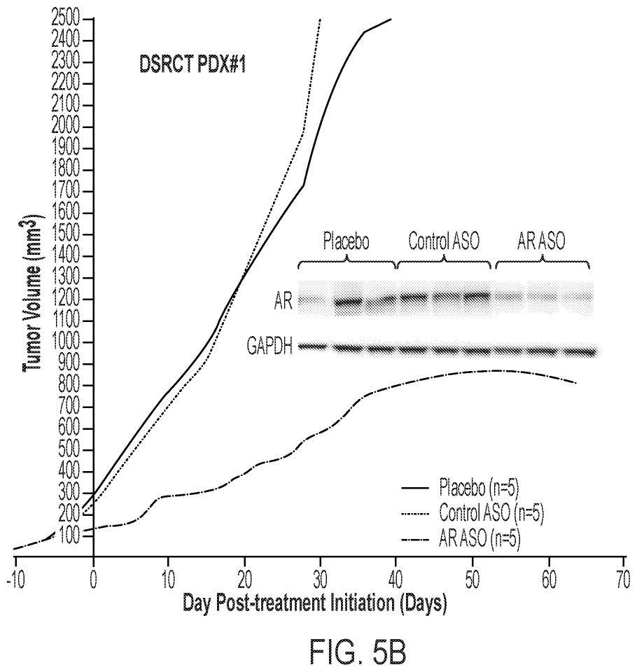

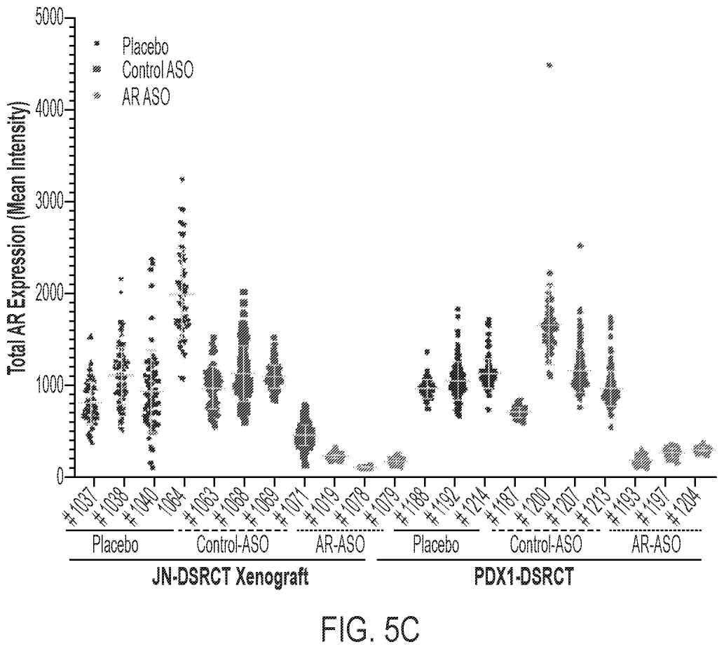

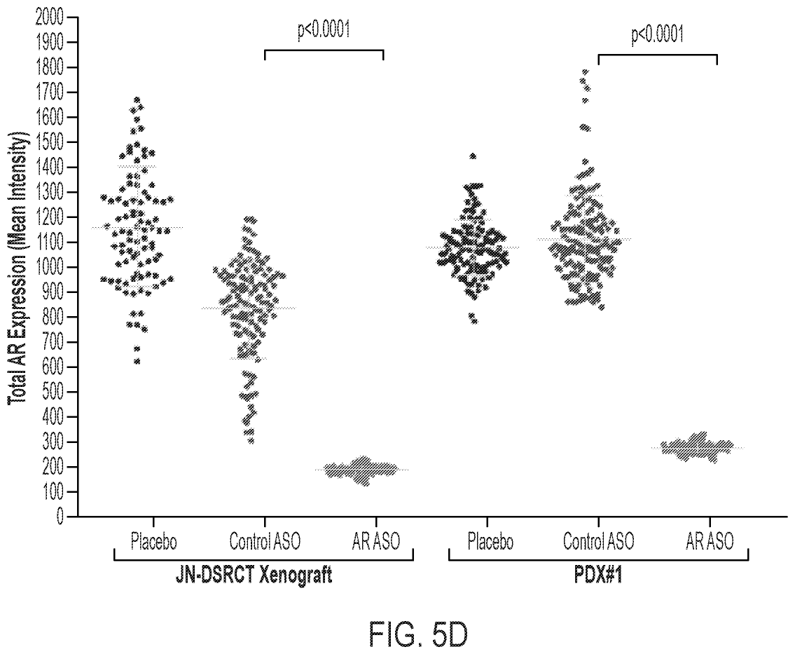

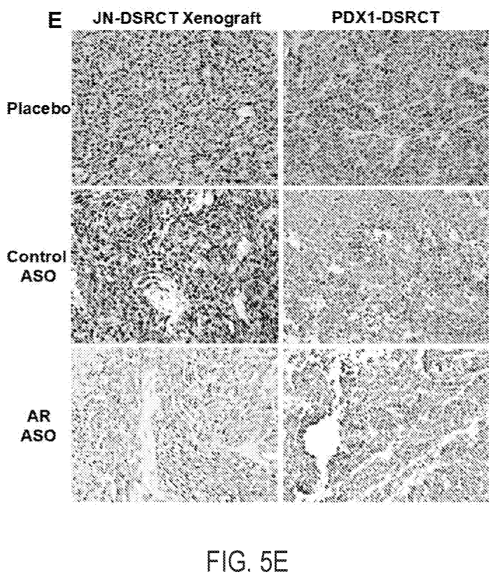

Finally, DSRCT patient-derived xenografts and JN-DSRCT tumor cells-bearing NSG immunocompromised mice were treated with anti-AR ASOs. It was observed that anti-AR ASO treated mice showed considerably reduced tumor burden and improved survival compared to mice in the placebo- and control ASO-treated groups (FIG. 5, p=0.0001 for PDX animal model and p=0.00097 for JN-DSRCT cell xenograft animal model).

In conclusion, proteomic profiling showed increased expression of AR in both DSRCT patients and cell line samples which distinguishes DSRCT patients from ES patients. Furthermore, AR stimulation enhanced in vitro cell proliferation, an effect that was mitigated using anti-AR targeted ASOs. Thus, AR can be targeted with anti-AR therapy, such as anti-AR targeted ASOs, for the treatment of small round cell tumors, such as DSCRT.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
P. H. Stahl & C. G. Wermuth eds., Verlag Helvetica Chimica Acta, 2002.
Remington's Pharmaceutical Sciences 22^{nd} edition, 2012.
U.S. Pat. No. 9,175,291
U.S. Pat. No. 9,567,588

What is claimed is:

1. A method for treating small round cell tumor in a subject comprising administering an effective amount of an anti-androgen receptor (anti-AR) therapy to the subject.

2. The method of claim 1, wherein the small round cell tumor is desmoplastic small round cell tumor (DSRCT).

3. The method of claim 1 or 2, wherein the subject is human.

4. The method of any of claims 1-3, wherein the anti-AR therapy comprises an AR antisense oligonucleotide.

5. The method of claim 4, wherein the AR antisense oligonucleotide comprises SEQ ID NO:1 (TTGATTTAATGGTTGC).

6. The method of claim 4, wherein the AR antisense oligonucleotide is AZD5312.

7. The method of any of claims 4-6, wherein the AR antisense oligonucleotide is administered intravenously.

8. The method of any of claims 4-7, wherein the AR antisense oligonucleotide is administered at a dose of 150-900 mg.

9. The method of any of claims 4-8, wherein the AR antisense oligonucleotide is administered more than once.

10. The method of any of claims 1-9, wherein the anti-AR therapy is an androgen receptor antagonist, androgen synthesis inhibitor, or an antigonadotropin.

11. The method of claim 10, wherein the androgen receptor antagonist is cyproterone acetate, megestrol acetate, chlormadinone acetate, spironolactone, oxendolone, flutamide, bicalutamide, nilutamide, topilutamide, enzalutamide, or apalutamide.

12. The method of claim 10, wherein the androgen synthesis inhibitor is ketoconazole, abiraterone acetate, seviteronel, aminoglutethimide, finasteride, dutasteride, epristeride, or alfatradiol.

13. The method of claim 10, wherein the antigonadotropin is leuprorelin, cetrorelix, allylestrenol, chlormadinone acetate, cyproterone acetate, gestonorone caproate, hydroxyprogesterone caproate, medroxyprogesterone acetate, megestrol acetate, or oxendolone.

14. The method of any of claims 1-13, wherein the anti-AR therapy is selected from the group consisting of enzalutamide (MDV3100), ARN-509, ODM-201, abiraterone acetate, Galeterone (TOK001), orteronel (TAK700) and VT464.

15. The method of any of claims 1-14, further comprising administering at least one additional anti-cancer therapy.

16. The method of claim 15, wherein the anti-cancer therapy is an anti-TAZ therapy.

17. The method of claim 15, wherein the anti-cancer therapy is an anti-EWSR1 therapy.

18. The method of claim 16, wherein the anti-TAZ therapy comprises an antisense oligonucleotide.

19. The method of claim 17, wherein the anti-EWSR1 therapy comprises an antisense oligonucleotide.

20. The method of claim 15, wherein the anti-cancer therapy is chemotherapy, immunotherapy, surgery, radiotherapy, or biotherapy.

21. The method of any of claims 15-20, wherein the anti-cancer therapy is administered orally, intravenously, intraperitoneally, intratracheally, intratumorally, intramuscularly, endoscopically, intralesionally, percutaneously, subcutaneously, topically, regionally, or by direct injection or perfusion.

22. The method of any of claims 15-21, wherein the anti-AR therapy and/or at least one additional anti-cancer therapy is administered simultaneously.

23. The method of any of claims 15-21, wherein the anti-AR therapy is administered prior to the at least one additional anti-cancer therapy.

24. A composition comprising an effective amount of an anti-AR therapy for use in the treatment of a small round cell tumor in a subject.

25. The composition of claim 24, wherein the small round cell tumor is DSRCT.

26. The composition of claim 24 or 25, wherein the anti-AR therapy comprises an AR antisense oligonucleotide.

27. The composition of claim 26, wherein the AR antisense oligonucleotide is AZD5312.

28. The use of a composition comprising an effective amount of an anti-AR therapy for the treatment of a small round cell tumor in a subject.

29. The use of claim 28, wherein the small round cell tumor is DSRCT.

30. The use of claim 28 or 29, wherein the anti-AR therapy comprises an AR antisense oligonucleotide.

31. The use of claim 30, wherein the AR antisense oligonucleotide is AZD5312.
